(19) United States
(12) Patent Application Publication
(10) Pub. No.: US 2024/0197532 A1
(43) Pub. Date: Jun. 20, 2024

(54) LASIK CORNEAL FLAP CUTTING PATTERNS FOR BUBBLE MANAGEMENT

(71) Applicant: AMO DEVELOPMENT, LLC, Irvine, CA (US)

(72) Inventors: Andrew Voorhees, Sunnyvale, CA (US); Hong Fu, Pleasanton, CA (US); James Hill, Santa Ana, CA (US); Mohammad Saidur Rahaman, San Jose, CA (US); Wenzhi Gao, Union City, CA (US); Brian Schwam, Jacksonville, FL (US); Paul Gray, San Jose, CA (US); Cynthia Villanueva, San Jose, CA (US); Deepali Mehta-Hurt, Newark, CA (US); Jesse Nelson, San Francisco, CA (US); Michal Laron, Milpitas, CA (US)

(21) Appl. No.: 18/545,930

(22) Filed: Dec. 19, 2023

Related U.S. Application Data
(60) Provisional application No. 63/476,364, filed on Dec. 20, 2022.

(51) Int. Cl. A61F 9/008

(52) U.S. Cl. CPC A61F 9/008; A61F 2009/00842; A61F 2009/00872; A61F 2009/00897

(57) ABSTRACT

A method implemented in an ophthalmic surgical laser system that employs a resonant scanner, scan line rotator, and XY- and Z-scanners, for forming a corneal flap in a patient's eye with improved bubble management during each step of the flap creation process. A pocket cut is formed first below bed level, followed by the bed connected to the pocket cut, then by a side cut extending from the bed to the anterior corneal surface. The pocket cut includes a pocket region located below the bed level and a ramp region connecting the pocket region to the bed. The bed is formed by a bed cut, including multiple overlapping parallel raster scan passes, and a ring cut. The side cut is formed by multiple side-cut layers at different depths which are joined together. All cuts are formed by scanning a laser scan line generated by the resonant scanner.

### CROSS-REFERENCE TO RELATED PATENT APPLICATIONS

This application claims priority to U.S. Provisional Application No. 63/476,364, filed on Dec. 20, 2022, which is hereby incorporated by reference in its entirety.

### BACKGROUND OF THE INVENTION

### Field of the Invention

This invention relates to ophthalmic laser surgeries, and in particular, it relates to flap cutting in LASIK (laser-assisted in situ keratomileusis) surgery using an ultrafast resonant scanning femtosecond laser.

### Description of Related Art

Femtosecond lasers are used to cut flaps in the corneal stroma as the first step of LASIK (laser-assisted in situ keratomileusis) surgeries. A flap is typically formed by a bed cut which is parallel to the anterior corneal surface and a vertical side cut around the periphery of the bed cut expect for an uncut hinge region.

When using femtosecond lasers to cut a flap in the corneal stroma as a part of a LASIK procedure, the interaction of the laser pulses with the tissue can sometimes create excessive gas bubbles which can interfere with the continued cutting of the tissue, creating tissue bridges and rough bed cut surfaces. For example, when forming a flap bed cut in the tissue, gas bubbles may be created by the laser during previous laser raster scans, and may block laser beam in the current scan pass. This could lead to a region of uncut tissue. Gas bubbles can also shadow the laser beam for the subsequent segments of the flap cut, creating tissue bridges.

Commonly owned U.S. Pat. Appl. Pub. No. 20220175581, entitled “LASIK Flap Cutting Patterns Including Intrastromal Pocket for Bubble Management,” describes “A method implemented in an ophthalmic surgical laser system that employs a resonant scanner, scan line rotator, and XY- and Z-scanners, for forming a corneal flap in a patient's eye with improved bubble management during each step of the flap creation process. A pocket cut is formed first below bed level, followed by the bed connected to the pocket cut, then by a side cut extending from the bed to the anterior corneal surface. The pocket cut includes a pocket region located below the bed level and a ramp region connecting the pocket region to the bed. The bed is formed by a hinge cut and a first ring cut at lower laser energies, followed by a bed cut and then a second ring cut, which ensures that any location in the flap bed is cut twice to minimize tissue adhesion. The side cut is formed by multiple side-cut layers at different depths which are joined together. All cuts are formed by scanning a laser scan line generated by the resonant scanner.” (Abstract.)

### SUMMARY

The present invention is directed to a method and related apparatus for cutting a corneal flap in LASIK surgery that improves the cutting procedure.

An object of the flap cutting procedure of the present invention is to ensure that the entire flap bed is cut, to improve the ease of flap lift, and to reduce the likelihood of tissue tags and opaque bubble layer.

Embodiments of this invention provide flap cutting patterns including intrastromal pockets that can be implemented with a resonant scanning femtosecond laser. These flap cutting patterns will allow for gas bubbles to collect and vent posterior to and outside of the flap bed cut and side cut.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a method implemented in an ophthalmic surgical laser system for incising a cornea of a patient's eye to form a corneal flap, the method including: controlling a laser delivery system of the ophthalmic surgical laser system to deliver a pulsed laser beam to the cornea; controlling a high frequency scanner of the ophthalmic surgical laser system to scan the pulsed laser beam back and forth to form a laser scan line; and controlling a scan line rotator, an XY-scanner and a Z-scanner of the ophthalmic surgical laser system to move the laser scan line in the cornea to form the corneal flap, including forming a bed of the flap and forming a side cut of the flap, wherein the bed is located in a horizontal plane at a first depth from an anterior corneal surface, the bed defining a hinge line, wherein the side cut extends from the bed upwards to the anterior corneal surface to form a side of the flap, the side cut surrounding an entire periphery of the bed except the hinge line, and wherein forming the bed of the flap consist of: forming a bed cut by scanning the laser scan line in successive overlapping parallel raster scan passes; and forming a single ring cut along a periphery of the bed except for an area of the hinge cut by scanning the laser scan line along a circumference of the bed, wherein the ring cut covers all areas of the bed not covered by the bed cut.

In another aspect, the present invention provides an ophthalmic surgical laser system, which includes: a laser delivery system configured to deliver a pulsed laser beam to a cornea of a patient's eye; a high frequency scanner configured to scan the pulsed laser beam back and forth at a predefined frequency to form a laser scan line; a scan line rotator configured to rotate an orientation of the laser scan line; an XY-scanner and a Z-scanner configured to move the laser scan line in lateral and depth directions; and a controller operatively coupled to and programmed to control the scan line rotator, the XY-scanner and the Z-scanner to scan the laser scan line in the cornea to form a corneal flap, including to form a bed of the flap and to form a side cut of the flap, wherein the bed is located in a horizontal plane at a first depth from an anterior corneal surface, the bed defining a hinge line, wherein the side cut extends from the bed upwards to the anterior corneal surface to form a side of the flap, the side cut surrounding an entire periphery of the bed except the hinge line, and wherein the bed of the flap consist of a bed cut formed by scanning the laser scan line in successive overlapping parallel raster scan passes, and a single ring cut along a periphery of the bed except for an area of the hinge cut formed by scanning the laser scan line along a circumference of the bed, wherein the ring cut covers all areas of the bed not covered by the bed cut.

It is to be understood that both the foregoing general description and the following detailed description are exemplary and explanatory and are intended to provide further explanation of the invention as claimed.

### BRIEF DESCRIPTION OF THE DRAWINGS

FIGS. 1A-1E schematically illustrate a flap procedure for forming a corneal flap according to an embodiment of the present invention.

FIG. 2 schematically illustrates a corneal flap according to another embodiment of the present invention.

FIGS. 3A-3C schematically illustrates overlapping scan sweeps when forming a side cut of the corneal flap according to embodiments of the present invention.

FIGS. 4A-4B schematically illustrate a previously disclosed flap procedure for forming a corneal flap.

FIG. 5 schematically illustrates raster scan sweeps formed using a fast-scan-slow-sweep scanning scheme implemented by an ultrafast raster scanning femtosecond laser.

FIGS. 6A and 6B schematically illustrate two exemplary ultrafast raster scanning femtosecond ophthalmic laser systems which may be used to implement embodiments of the present invention.

### DETAILED DESCRIPTION OF PREFERRED EMBODIMENTS

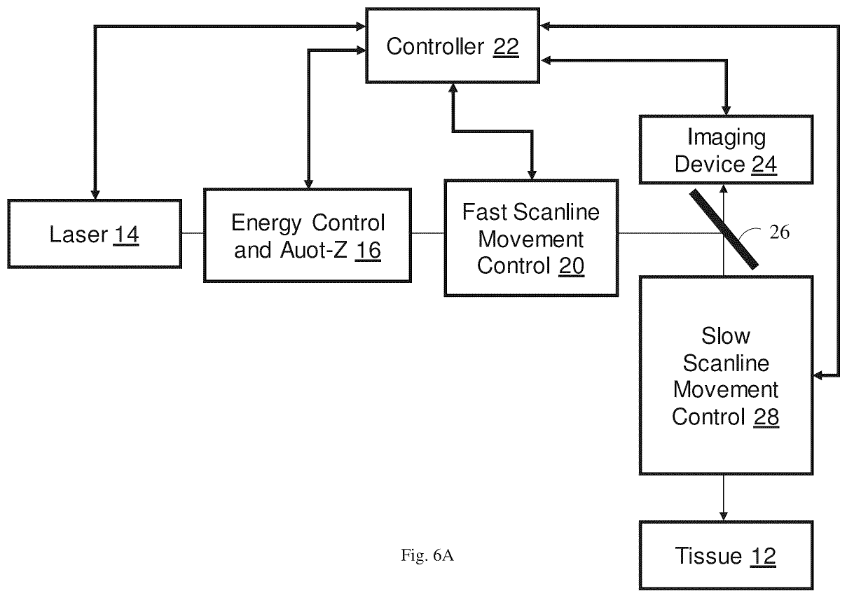

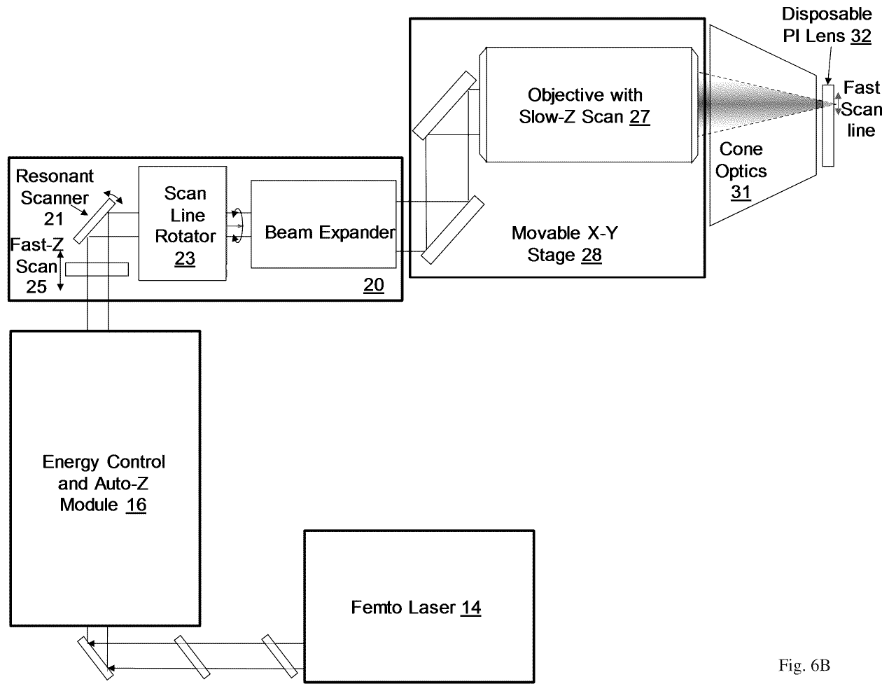

FIGS. 6A and 6B illustrate ultrafast raster scanning femtosecond ophthalmic laser systems that may be used to implement the flap formation procedures according to various embodiments of the present invention.

FIG. 6A shows a system 10 for making an incision in a tissue 12 of a patient's eye, such as a cornea. The system 10 includes, but is not limited to, a laser 14 capable of generating a pulsed laser beam, an energy control module 16 for varying the pulse energy of the pulsed laser beam, a fast scan line movement control module 20 for generating the scan line of the pulsed laser beam, a controller 22, and a slow scan line movement control module 28 for moving the laser scan line and delivering it to the tissue 12. The controller 22, such as a processor operating suitable control software, is operatively coupled with the fast scan line movement control module 20, the slow scan line movement control module 28, and the energy control module 16 to direct the scan line of the pulsed laser beam along a scan pattern on or in the tissue 12. In this embodiment, the system 10 further includes a beam splitter 26 and a imaging device 24 coupled to the controller 22 for a feedback control mechanism (not shown) of the pulsed laser beam. Other feedback methods may also be used. In an embodiment, the pattern of pulses may be summarized in machine readable data of tangible storage media in the form of a treatment table. The treatment table may be adjusted according to feedback input into the controller 22 from an automated image analysis system in response to feedback data provided from a monitoring system feedback system (not shown).

Laser 14 may comprise a femtosecond laser capable of providing pulsed laser beams, which may be used in optical procedures, such as localized photodisruption (e.g., laser induced optical breakdown). Localized photodisruptions can be placed at or below the surface of the tissue or other material to produce high-precision material processing. For example, a micro-optics scanning system may be used to scan the pulsed laser beam to produce an incision in the material, create a flap of the material, create a pocket within the material, form removable structures of the material, and the like. The term “scan” or “scanning” refers to the movement of the focal point of the pulsed laser beam along a desired path or in a desired pattern.

In other embodiments, the laser 14 may comprise a laser source configured to deliver an ultraviolet laser beam comprising a plurality of ultraviolet laser pulses capable of photodecomposing one or more intraocular targets within the eye.

Although the laser system 10 may be used to photoalter a variety of materials (e.g., organic, inorganic, or a combination thereof), the laser system 10 is suitable for ophthalmic applications in some embodiments. In these cases, the focusing optics direct the pulsed laser beam toward an eye (for example, onto or into a cornea) for plasma mediated (for example, non-UV) photoablation of superficial tissue, or into the stroma of the cornea for intrastromal photodisruption of tissue. In these embodiments, the surgical laser system 10 may also include a lens to change the shape (for example, flatten or curve) of the cornea prior to scanning the pulsed laser beam toward the eye.

FIG. 6B shows another exemplary diagram of the laser system 10. FIG. 6B shows components of a laser delivery system including a moveable XY-scanner (or movable XY-stage) 28 of a miniaturized femtosecond laser system. In this embodiment, the system 10 uses a femtosecond oscillator, or a fiber oscillator-based low energy laser. This allows the laser to be made much smaller. The laser-tissue interaction is in the low-density-plasma mode. An exemplary set of laser parameters for such lasers include pulse energy in the 40-100 nJ range and pulse repetitive rates (or “rep rates”) in the 2-40 MHz range, or more preferably, above 5 MHz. A fast-Z scanner 25 and a resonant scanner 21 direct the laser beam to a scan line rotator 23. When used in an ophthalmic procedure, the system 10 also includes a patient interface design that has a fixed cone nose 31 and a contact lens 32 that engages with the patient's eye. A beam splitter may be placed inside the cone 31 of the patient interface to allow the whole eye to be imaged via visualization optics. In some embodiments, the system 10 may use: optics with a 0.6 numerical aperture (NA) which would produce 1.1 μm Full Width at Half Maximum (FWHM) focus spot size; and a resonant scanner 21 that produces 0.2-1.2 mm scan line with the XY-scanner scanning the resonant scan line to a 1.0 mm field. The scan line rotator 23 (e.g., a Dove or Pechan prism, a set of mirrors, or the like, mounted on a rotating stage) rotates the resonant scan line to any direction on the XY plane. The fast-Z scanner 25 sets the incision depth. The slow scan line movement control module employs a movable XY-stage 28 carrying an objective lens with Z-scanning capability 27, referred to as slow-Z scanner because it is slower than the fast-Z scanner 25. The movable XY-stage 28 moves the objective lens to achieve scanning of the laser scan line in the X and Y directions. The objective lens changes the depth of the laser scan line in the tissue. The energy control and auto-Z module 16 may include appropriate components to control the laser pulse energy, including attenuators, etc. It may also include an auto-Z module which employs a confocal or non-confocal imaging system to provide a depth reference. The miniaturized femtosecond laser system 10 may be a desktop system so that the patient sits upright while being under treatment. This eliminates the need of certain opto-mechanical arm mechanism(s), and greatly reduces the complexity, size, and weight of the laser system. Alternatively, the miniaturized laser system may be designed as a conventional femtosecond laser system, where the patient is treated while lying down.

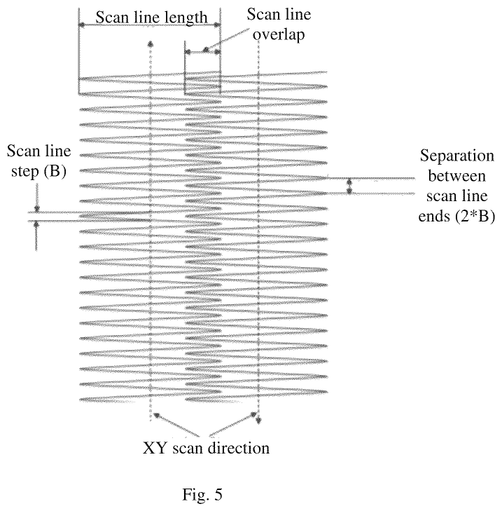

In preferred embodiments, the beam scanning can be realized with a “fast-scan-slow-sweep” scanning scheme, also referred herein as a fast-scan line scheme. The scheme consists of two scanning mechanisms: first, a high frequency fast scanner (e.g., the resonant scanner 21 of FIG. 6B) is used to scan the beam back and forth to produce a short, fast scan line; second, the fast scan line is slowly swept by much slower X, Y, and Z scan mechanisms (e.g. the moveable X-Y stage 28 and the objective lens with slow-Z scan 27, and the fast-Z scanner 25). For example, the laser system may use an 8 kHz (e.g. between 7 kHz and 9 kHz, or more generally, between 0.5 kHz and 20 kHz, or greater than 1 kHz) resonant scanner 21 to produce a fast scan line of about 1 mm (e.g., between 0.9 mm and 1.1 mm, or more generally, between 0.2 mm and 1.2 mm) and a scan speed of about 25 m/sec, and X, Y, and Z scan mechanisms with the scan speed (sweeping speed) smaller than about 0.1 m/sec. The fast scan line may be perpendicular to the optical beam propagation direction, i.e., it is always parallel to the XY plane. The trajectory of the slow sweep can be any three dimensional curve drawn by the X, Y, and Z scanning devices (e.g., XY-scanner 28 and fast-Z scanner 25). FIG. 5 schematically illustrates two parallel, overlapping scan sweeps formed by the fast-scan-slow-sweep scanning scheme.

An advantage of the “fast-scan-slow-sweep” scanning scheme is that it only uses small field optics (e.g., a field diameter of 1.5 mm) which can achieve high focus quality at relatively low cost. The large surgical field (e.g., a field diameter of 10 mm or greater) is achieved with the XY-scanner, which may be unlimited.

As described in more detail below, the flap procedures in various embodiments of the present invention utilize the fast-scan-slow-sweep scanning scheme to form various cuts of the flap. The controller of the laser system controls the various components of the system to form various cuts described below.

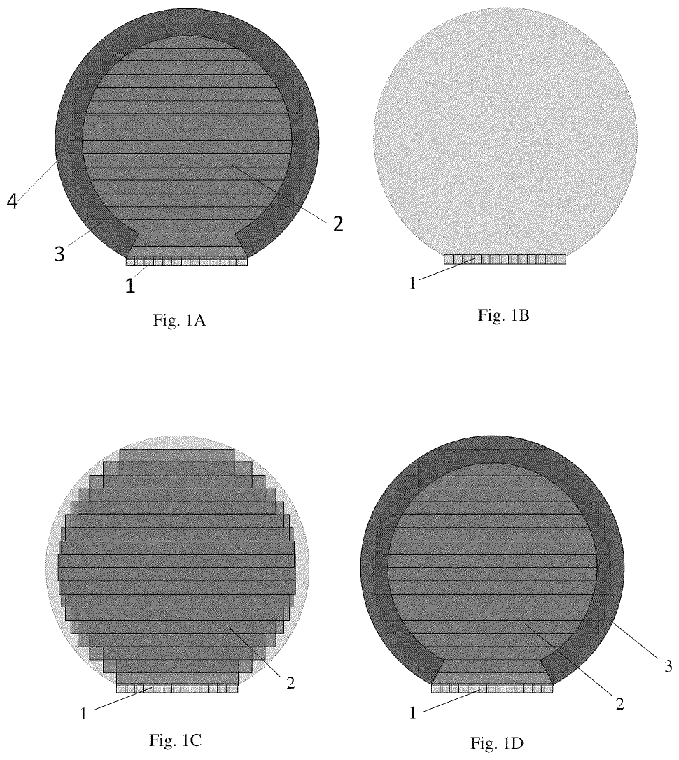

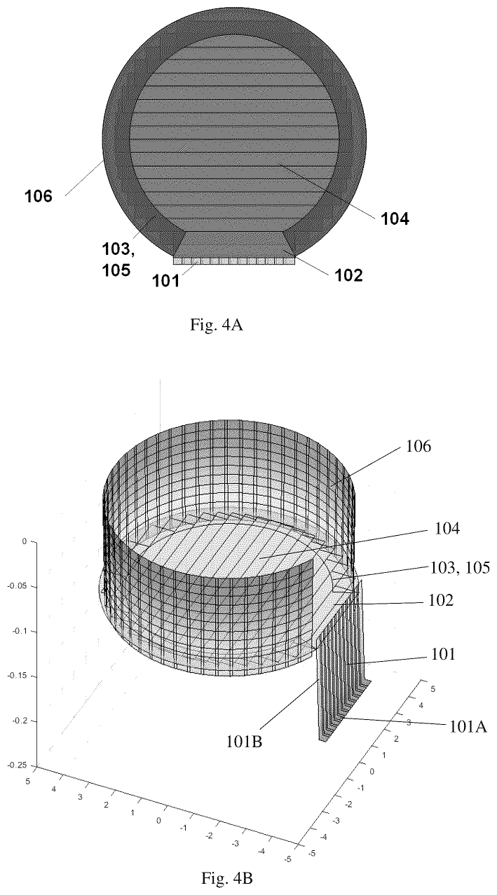

A previously disclosed technique for managing gas bubble formation during bed cut for a corneal flap, described in the above-mentioned U.S. Pat. Appl. Pub. No. 20220175581 (“the '581 publication”), is shown in FIGS. 4A (top view) and 4B (perspective view), adapted from FIGS. 1A-1B of the '581 publication. The flap procedure includes the following components, in the cutting order: a pocket cut 101 with a ramp, a low energy hinge cut 102, a first (low energy) ring cut 103, a bed cut 104, a second (normal energy) ring cut 105, and a side cut 106. The hinge cut 102, ring cuts 103 and 105, and bed cut 104 are located on the same plane, parallel to the anterior corneal surface, to form the bed of the flap. The overall shape of the bed is a circular area except for an uncut arc segment serving as the hinge portion of the flap (the straight line of the arc segment may be referred to as the hinge line). The side cut 106, which extends from the bed upwards to the anterior corneal surface to form the side of the flap, is located around the entire periphery of the bed except for an uncut arc at the hinge. The pocket cut 101 is located at the hinge line of the bed and extends below the bed level.

The pocket cut 101 includes a pocket region 101A and a ramp region 101B. The ramp region 101B is aligned along the hinge line of the flap in the top view, and extends from a depth slightly above the bed level (e.g., 2 to 20 μm above the bed level) to a depth about 70 to 150 μm below the bed level. The pocket region 101A extends substantially horizontally from the lower end of the ramp region 101B for about 150 to 400 μm. The pocket cut 101 preferably extends along the entire length of the hinge line, or along a part of the length of the hinge line. The ramp region 101B may be vertical, or alternatively be inclined (e.g., within 0-170 degrees from the vertical direction). Overall, the pocket cut 101 has a rectangular shape when viewed from the top, and also has a rectangular shape in a side view when viewed along a direction perpendicular to the hinge line.

The pocket cut 101 is made first, with the ramp region connecting to the bed level of the bed cut to be formed, along the hinge line. The pocket cut may be made by placing the laser scan line parallel to the hinge line and scanning the scan line along the intended surface of the pocket cut using the XY scanner and the Z scanner. The scanning direction is radially inwardly for the pocket region 101A and in a deep-to-shallow direction for the ramp region 101B. Multiple passes may be executed side-by-side (preferably with edge overlaps) to form the entire pocket.

Then, a cutting pass is made at the bed level, located across the hinge portion along the hinge line, forming the hinge cut 102 which connects to the pocket cut. A low energy ring cut 103 is made at the bed level along the periphery of the bed circle except for the hinge portion, to create the peripheral edge of the flap bed. The ring cut 103 is made by placing the laser scan line in a radial direction at the required distance from the center of the bed, so that its outer end is located at the circumference of the bed circle, and scanning the scan line using the XY scanner along the circumferential direction while using the scan line rotator to rotate the scan line direction to keep it in the radial direction. The order of the hinge cut 102 and ring cut 103 may be reversed. Both cuts are made at laser pulse energy levels that are lower than the other cutting steps, e.g., at 90% (or more generally, from 85% to 95%) of the pulse energy of the other cutting steps. These lower energy cuts generate less bubbles and less distortion of the tissue. In preferred embodiments, the pulse energy for the pocket cut, the bed cut, the second ring cut, and the side cut is 40 to 90 nJ.

Then, the bed cut 104 is made by creating overlapping parallel raster scan passes of the laser scan line, covering substantially the entire bed circle (at least all the areas not covered by the ring cut 103) except for the arc segment of the hinge. Preferably, adjacent parallel raster scan passes overlap with each other in the width direction by at least 50% of their widths, so that in effect, each point is covered by at least two passes. Note that one of the scan passes preferably overlaps with the low energy hinge cut 102. The second ring cut 105 is then made in the same area of the first ring cut 103 to ensure tissue separation at the edges of the bed. The second ring cut 105 is made in the same way as the first ring cut 103 but at the normal pulse energy.

The hinge cut 102, first ring cut 103, bed cut 104, and second ring cut 105 overlap each other so that any given point within the bed is covered by at least two passes, which minimize residual uncut tissue bridges. Moreover, the cutting sequence of the hinge cut 102, first ring cut 103, bed cut 104, and second ring cut 105 ensures that a venting channel is always present that connects the current cutting point through earlier-formed cuts to the pocket, so that the gas formed at the current cutting point always has somewhere to escape to, thereby voiding opaque bubble layer formation.

Lastly, a side cut 106 is made. The side cut may be formed by placing the laser scan line along the circle of the side cut in a tangential direction, and scanning the scan line in the vertical or near vertical direction using the Z scanner (and optionally the XY scanner) of the ophthalmic laser system. After each vertical scan, the scan line is moved by the XY scanner to the next position along the circle and rotated by the scan line rotator to be again tangent to the circle, and the vertical scanning is repeated. In alternative embodiments, the side cut 106 is formed in using a multilayer technique, described in more detail later.

The cutting order described above, which cuts the ring cut and hinge cut twice, the first time at lower energy, has the advantage of avoiding tissue bridges. The low energy hinge cuts 102 and low energy ring cut 103 generate less bubbles and less distortion of tissue.

A corneal flap procedure for forming a corneal flap according to an embodiment of the present invention is described now with reference to FIGS. 1A-1E. As shown in FIGS. 1A (top view) and 1E (perspective view), the flap procedure includes the following components, in the cutting order: a pocket cut 1 with a ramp, a bed cut 2, a ring cut 3, and a side cut 4. The bed cut 2 and ring cut 3 are located on the same plane, parallel to the anterior corneal surface, to form the bed of the flap. The overall shape of the bed is a circular area except for an uncut arc segment serving as the hinge portion of the flap (the straight line of the arc segment may be referred to as the hinge line). The pocket cut 1 is located at the hinge line of the bed and extends below the bed level. The side cut 4, which extends from the bed upwards to the anterior corneal surface to form the side of the flap, is located around the entire periphery of the bed except for an uncut arc at the hinge. The side cut 4 may be vertical (perpendicular to the bed) or non-vertical. Preferably, the side cut 4 forms an angle of 30° to 150° with respect to the bed.

The pocket cut 1 includes a pocket region 1A and a ramp region 1B. In this example, the ramp region 1B extends substantially vertically parallel to the hinge line of the flap, but shifted slightly from the hinge line toward the bed center in the top view. The ramp region 1B extends from a depth at the bed level to a depth about 70 to 150 μm, preferably about 100 μm, below the bed level. The pocket region 1A extends substantially horizontally from the lower end of the ramp region 1B for about 150 to 400 μm, preferably about 200 μm. The pocket cut 1 preferably extends along the entire length of the hinge line, or along a part of the length of the hinge line. Although the ramp region 1B is substantially vertical in the illustrated embodiment, it may alternatively be non-vertical (e.g., within 0-170 degrees from the vertical direction). Overall, the pocket cut 1 has a rectangular shape when viewed from the top, and also has a rectangular shape in a side view when viewed along a direction perpendicular to the hinge line.

To form the flap, the pocket cut 1 is made first (FIG. 1B), with the ramp region reaching the bed level of the bed cut to be formed near the hinge line (the circle in FIG. 1B depicts the bed to be formed). The pocket cut 1 may be made by placing the laser scan line parallel to the hinge line at the pocket depth and scanning the scan line along the intended surface of the pocket cut using the XY scanner and the Z scanner of the ophthalmic laser system. The scanning direction is first radially inwardly for the pocket region 1A and then in a deep-to-shallow direction for the ramp region 1B. Multiple passes may be executed side-by-side (preferably with edge overlaps) to form the entire pocket spanning the length of the hinge. The pocket cut 1 is optional.

Next, the bed cut 2 is made (FIG. 1C) by creating overlapping parallel raster scan passes of the laser scan line, covering substantially the entire bed circle except for the arc segment of the hinge. Preferably, adjacent parallel raster scan passes (represented by the rectangular shapes in FIG. 1C) overlap with each other in the width direction by 20 to 80% of their widths. In some embodiments, the overlap is at least 50%, so that in effect, each point is covered by at least two passes. The raster scan passes are preferably parallel to the hinge line, with the first pass located along the hinge line and having the same length as the pocket, and the other passes sequentially shifted away from the hinge line.

The ring cut 3 is made next along the periphery of the bed circle except for the hinge portion (FIG. 1D), to create the peripheral edge of the flap bed. The two ends of the ring cut 3 are preferably located at the edge of the pocket cut 1. The ring cut 3 covers the jagged edges resulting from the multiple passes of the bed cut, and ensures tissue separation at the circular edge of the flap bed. The ring cut 3 is made by placing the laser scan line in a radial direction at the required distance from the center of the bed, so that its outer end is located at the circumference of the bed circle, and scanning the scan line using the XY scanner along the circumferential direction while using the scan line rotator to rotate the scan line direction to keep it in the radial direction.

Lastly, the side cut 4 is made (FIG. 1A). In some embodiments, the side cut may be formed by placing the laser scan line along the circle of the side cut in a tangential direction, and scanning the scan line in the vertical (or non-vertical) direction using the Z scanner (or both the Z scanner and the XY scanner). After each scan, the scan line is moved by the XY and Z scanners to the next position along the circle and rotated by the scan line rotator to be again tangent to the circle, and the scanning is repeated. In alternative embodiment, the side cut 4 is formed using a multilayer technique, described in more detail later.

In preferred embodiments, the pulse energy for forming the pocket, bed, ring and side cuts is less than 100 nJ, or more preferably, from 40 to 90 nJ, and the pulse energy of the bed cut and the side cut may be set independently. The scan line length is between 400 and 1100 μm. In preferred embodiments, the speed of the XY and Z movements of the scan line is such that the distance traversed during a single period of the high frequency scanner is less than 6 μm (see FIG. 5, where the distance traversed during a single period of the high frequency scanner is 2*B).

Note that although in FIG. 1E the ramp region 1B is shown as extending slightly above the bed level and intersecting the bed along a line parallel to the hinge line but closer to the bed center, in alternative embodiments, the ramp region may terminate at the bed level and/or intersect the bed at the hinge line.

Although in FIGS. 1A-1E the shape of the bed is a circle except for the hinge portion, in alternative embodiments, the bed may be an elliptical shape except for the hinge portion.

Compared to the cutting procedure described in the '581 publication (FIGS. 4A-4B), in the above-described embodiment of the present invention (FIGS. 1A-1E), a low energy hinge cut and a first low energy ring cut are not performed; the bed is formed by only the bed cut 2 and the single ring cut 3, performed at the same pulse energy. In the above embodiment, the execution sequence for the various cuts ensures that the first pass of the bed intersects the entire pocket ramp, thus creating a wider and easier path for gas to flow away from the subsequent bed cut scans. Further the overlap of the subsequent parallel bed scans continues to allow the gas to have an easy path to the pocket. This is an improvement over the prior pattern described in FIGS. 4A-4B where the initial portion of the bed is the ring cut which only intersects a small portion of the pocket ramp and all gas must move back to the pocket through a smaller entry. The above embodiment significantly reduces the likelihood that opaque bubble layer will interfere with the formation of the bed cut. In a clinical validation study, the cutting procedure according to the embodiment of the present invention provided significantly improved ease of flap lift as compared to the cutting procedure described in the '581 publication. It also reduces the number of cutting steps and reduces the procedure time.

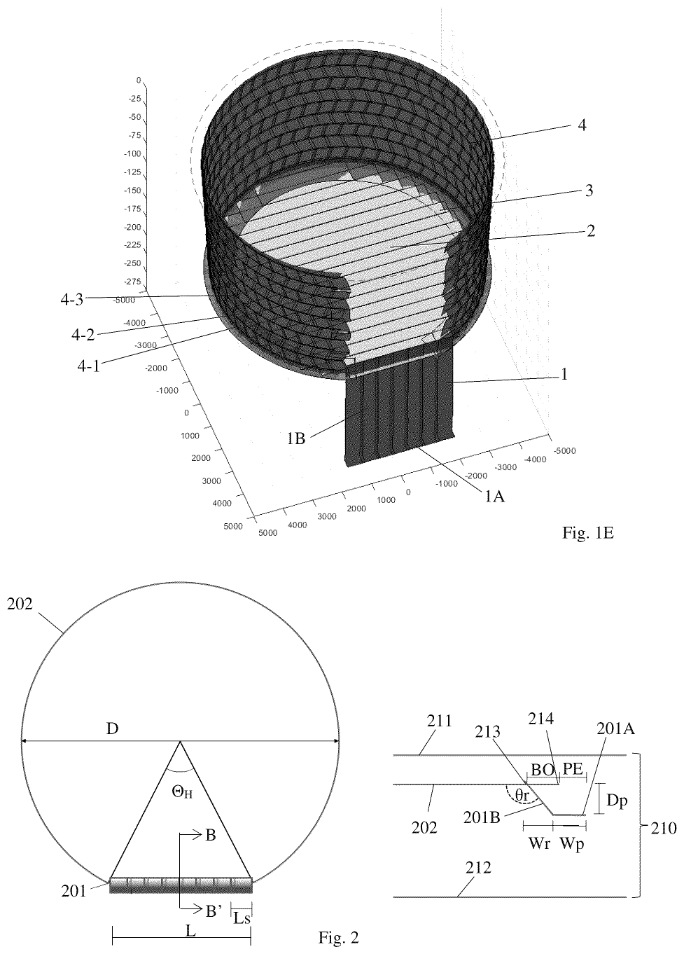

FIG. 2 (top view and depth profile) schematically illustrates a flap procedure that forms a corneal flap with a pocket cut according to another embodiment of the present invention. As shown in FIG. 2, the flap is formed of a bed cut 202, which is a circular or elliptical area except for an uncut arc segment serving as a hinge portion, a pocket cut 201 connected to the bed cut near the hinge line, including a ramp region 201B and a pocket region 201A, and a side cut (also designated by the reference symbol 202) located around the periphery of the bed cut except for the uncut hinge portion. The pocket cut 201 (the ramp region 201B and the pocket region 201A collectively) has a rectangular shape in the top view, with a length L approximately equal to the length of the hinge portion.

FIG. 2 also illustrates a depth profile 210 through the center of the hinge as indicated by arrows B-B′, showing the anterior corneal surface 211, posterior corneal surface 212, the ramp region 201B, the pocket region 201A, and a part of the bed cut 202. The depth profile is the same throughout the entire length L of the pocket cut 201. As seen in the depth profile, the pocket region 201A is located below (deeper than) the bed cut 202, the upper-inner end of the non-vertical ramp region 201B is connected to the bed cut 201 at a location 213 that is offset from the hinge line 214 toward the bed center, and the lower-outer end of the ramp region 201B is connected to the pocket region 201A. This way, the ramp region 201B connects the pocket region 201A to the bed cut 202. In the illustrated embodiments, the depth profile of the ramp 202 is linear, but it may alternatively have a curved shape. For example, it may have rounded connections with the pocket region 201A. As shown in FIG. 2, the portion of the bed cut 202 that extends beyond the intersection location 213 may be referred to as “bed overcut” (BO), and the portion of the pocket cut 201 that extends beyond the hinge line 214 (in the top view) may be referred to as “pocket extension” (PE).

The various cuts of the embodiment of FIG. 2 are formed in similar manners as those of the embodiment of FIG. 1A-1D.

In one particular example (see FIG. 2), the flap diameter D is 9.0 mm, the pocket width Wp is 200 μm, the pocket depth Dp (measured from the bed level) is 100 μm, the ramp angle θr relative to the horizontal plane is 150°, the ramp width Wr is 170 μm, the horizontal bed overcut BO is 250 μm, and the pocket extension PE over the hinge line is 120 μm. The hinge length is 4.2 mm, and the hinge angle Θₙ (the angle spanned by the hinge line with respect to the bed center) is approximately 55°. The scan line length Ls for forming the pocket cut 201 is about 600 μm, and eight parallel scans are used in this example to form the entire length of the pocket cut 201. Other appropriate parameters may be used. More generally, the flap diameter D may be between 5 and 10 mm, the pocket width Wp may be between 150 and 400 μm, and the hinge angle Θ_{H} may be between 45 and 90 degrees.

Pocket cuts of various other shaped pocket cut may be formed, some of which are described in the above-mentioned '581 publication, which is incorporated herein by reference in its entirety.

In the flap cutting procedures according to the above embodiments, the pockets allow the gas bubbles to move into the pockets and out of the laser path of the subsequent cut. It is not essential for the bubbles to move all the way back to the pocket, however, so long as they move backward to earlier cut areas. Thus, for example, in a bed cut, ring cut, side cut cutting order, when cutting the side cut, it is not essential for the gas bubbles to move into the pocket; rather, it is sufficient if they move back to the ring or bed cut.

Referring back to FIG. 1E, in a preferred embodiment, the side cut 4 is formed using a multilayer technique to form a layered side cut. More specifically, the side cut 4 is formed one horizontal layer at a time, where each layer 4-1, 4-2, 4-3, etc. is a part of the overall side cut but extends only in a depth range that is a sub-range of the entire depth range of the side cut. All but the top layer are completely located within the cornea and do not reach the anterior corneal surface. The layers are aligned with each other along the entire depth range and connect with each other to form the side cut. The adjacent layers preferably overlap each other in the depth direction (i.e., their sub-ranges overlap each other). In one example, the layer thickness, i.e. the size of the depth sub-range, is about 25 μm, and the overlap between two adjacent layers is about 10 μm. More generally, the layer thickness may be from 10 to 40 μm, and the layer overlap may be from 2 to 15 μm. The bottom-most layer may extend below the bed level and the top-most layer may extend above the anterior corneal surface, to ensure proper separation of the flap. The number of side cut layers is determined by the total depth of the side cut, the thickness of each layer and the amount of overlap between adjacent layers. The example illustrated in FIG. 1E has ten layers. The different layers may also have different thicknesses.

The side cut layers are formed in a sequence from posterior to anterior (i.e. deeper to shallower relative to the anterior corneal surface), preferably changing layers at the corner of the hinge to start the subsequent side cut layer. The top of the last side cut layer that is located completely inside the cornea is preferably within less than 20 μm from the top surface of epithelium in order to manage the bubbles generated within the ring cut, bed cut and side cut.

More specifically, each side cut layer 4-1, etc. is created by placing the laser scan line tangent to the side-cut path (along the circumference), oscillating it sinusoidally in the Z direction, simultaneously moving it around the side-cut path in the circumferential XY direction, and simultaneously rotating the scan line to keep it tangent to the side-cut path. In one particular example, the Z oscillation frequency is 120 Hz, the scan line length is 600 μm, the scan line overlap is 25%, the glass overcut at the anterior corneal surface is 90 μm, the side cut angle is 120°, and the flap diameter was 8.0 mm.

Preferably, during the anterior-to-posterior half of each sinusoidal period, the laser beam is blanked using a fast blanking technique in order to manage the generated bubbles in the corneal tissue. For example, fast blanking may be achieved by temporarily reducing the laser pulse energy to below the photodisruption threshold of the corneal tissue and increasing pulse duration so that no tissue cutting occurs, which in turn may be achieved by temporarily increasing the laser pulse repetition rate while keeping the pump current of the laser constant. Thus, each posterior-to-anterior half period form a scan sweep.

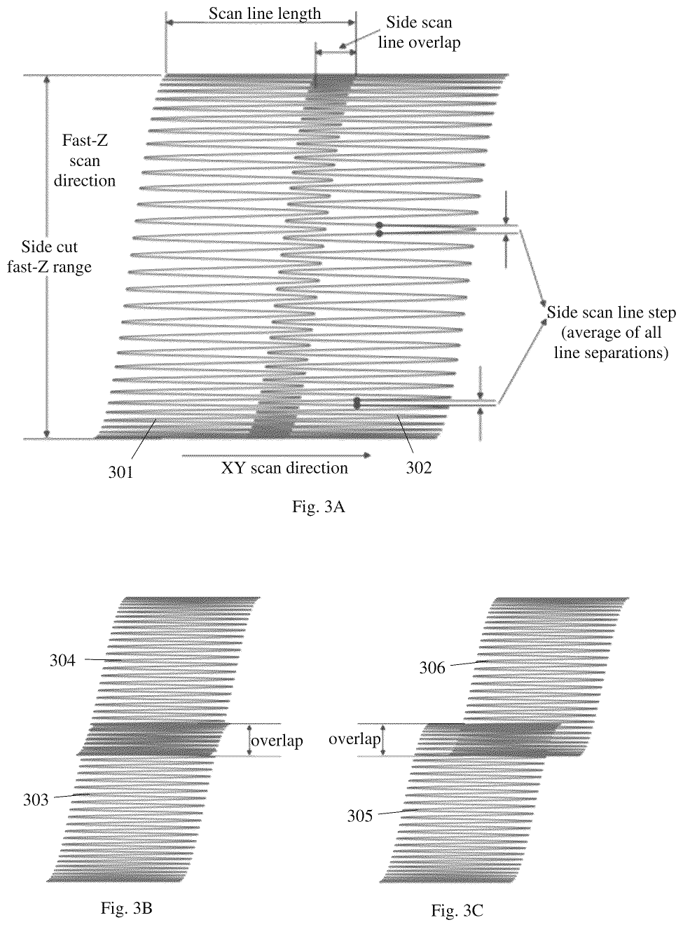

FIG. 3A schematically illustrates two adjacent posterior-to-anterior scan sweeps 301, 302 that form a part of a side cut layer, the two sweeps overlapping each other in the XY scan (horizontal) direction at their edges. FIG. 3B schematically illustrates an example of two scan sweeps 303, 304 in two adjacent side cut layers that overlap each other in the Z (depth) direction. FIG. 3C schematically illustrates an example of two scan sweeps 305, 306 in two adjacent side cut layers that overlap each other in the Z (depth) direction; the two scan sweeps in this example are also shifted relative to each other in the XY scan direction.

It will be apparent to those skilled in the art that various modification and variations can be made in the corneal flap procedure and related apparatus of the present invention without departing from the spirit or scope of the invention. Thus, it is intended that the present invention cover modifications and variations that come within the scope of the appended claims and their equivalents.

What is claimed is:

1. A method implemented in an ophthalmic surgical laser system for incising a cornea of a patient's eye to form a corneal flap, the method comprising:
controlling a laser delivery system of the ophthalmic surgical laser system to deliver a pulsed laser beam to the cornea;
controlling a high frequency scanner of the ophthalmic surgical laser system to scan the pulsed laser beam back and forth to form a laser scan line; and
controlling a scan line rotator, an XY-scanner and a Z-scanner of the ophthalmic surgical laser system to move the laser scan line in the cornea to form the corneal flap, including forming a bed of the flap and forming a side cut of the flap,
wherein the bed is located in a horizontal plane at a first depth from an anterior corneal surface, the bed defining a hinge line, wherein the side cut extends from the bed upwards to the anterior corneal surface to form a side of the flap, the side cut surrounding an entire periphery of the bed except the hinge line, and
wherein forming the bed of the flap consists of:
forming a bed cut by scanning the laser scan line in successive overlapping parallel raster scan passes; and
forming a single ring cut along a periphery of the bed except for an area of the hinge cut by scanning the laser scan line along a circumference of the bed, wherein the ring cut covers all areas of the bed not covered by the bed cut.

2. The method of claim 1, wherein a laser pulse energy of the bed cut and a laser pulse energy of the side cut are set independently.

3. The method of claim 1, wherein each successive one of the raster scan passes overlaps a previous scan pass by 20 to 80% of a length of the scan lines.

4. The method of claim 1, wherein the step of forming the side cut includes forming a plurality of side cut layers in a sequence, each side cut layer extending within a depth range relative to the anterior corneal surface, wherein all except one of the plurality of side cut layers are located entirely within the cornea without reaching the anterior corneal surface, and wherein the plurality of side cut layers are aligned with each other and connect with each other to form the side cut.

5. The method of claim 4, wherein each of the plurality of side cut layers is formed by placing the laser scan line tangent to a circumference of the side cut, moving the laser scan line in a vertical direction, simultaneously moving the laser scan line around the circumference, and simultaneously rotating the scan line to keep it tangent to the circumference.

6. The method of claim 4, wherein adjacent side cut layers overlap each other in a depth direction.

7. The method of claim 1, wherein the side cut forms a non-90 degree angle relative to the bed cut.

8. The method of claim 7, wherein the side cut forms an angle of from 30° to 150° relative to the bed cut.

9. The method of claim 1, further comprising, prior to forming the bed cut, controlling the scan line rotator, the XY-scanner and the Z-scanner of the ophthalmic surgical laser system to move the laser scan line in the cornea to form a pocket cut,
wherein the pocket cut extends from a second depth posterior to the bed to the depth of the bed, wherein the bed cut is connected to the pocket cut;
wherein the pocket cut includes a ramp region and a pocket region connected to each other, wherein the pocket region is located at the second depth, and wherein the ramp region extends between the first depth and the second depth and is connected to both the bed and the pocket region.

10. The method of claim 1, wherein a repetition rate of the laser is greater than 5 MHz.

11. The method of claim 1, wherein a frequency of the high frequency scanner is greater than 1 kHz.

12. The method of claim 1, wherein a pulse energy of the pulsed laser is less than 100 nJ.

13. The method of claim 1, wherein a length of the scan line in the bed cut, the ring cut and the side cut is between 400 and 1100 μm.

14. The method of claim 1, wherein the controller controls the XY-scanner and the Z-scanner are controlled to move the scan line such that a distance traversed during a single period of the high frequency scanner is less than 6 μm.

15. The method of claim 1, wherein the flap has an elliptical shape.

16. The method of claim 1, further comprising, during transitions between successive scan passes of the bed and side cut, temporarily blanking pulses of the pulsed laser beam by temporarily increasing a pulse repetition rate of the pulsed laser beam which temporarily reduces the pulse energy of the pulsed laser beam to below a photodisruption threshold of a corneal tissue and increases the pulse duration.

17. An ophthalmic surgical laser system, comprising:
a laser delivery system configured to deliver a pulsed laser beam to a cornea of a patient's eye;
a high frequency scanner configured to scan the pulsed laser beam back and forth at a predefined frequency to form a laser scan line;
a scan line rotator configured to rotate an orientation of the laser scan line;
an XY-scanner and a Z-scanner configured to move the laser scan line in lateral and depth directions; and
a controller operatively coupled to and programmed to control the scan line rotator, the XY-scanner and the Z-scanner to scan the laser scan line in the cornea to form a corneal flap, including to form a bed of the flap and to form a side cut of the flap,
wherein the bed is located in a horizontal plane at a first depth from an anterior corneal surface, the bed defining a hinge line, wherein the side cut extends from the bed upwards to the anterior corneal surface to form a side of the flap, the side cut surrounding an entire periphery of the bed except the hinge line, and
wherein the bed of the flap consist of a bed cut formed by scanning the laser scan line in successive overlapping parallel raster scan passes, and a single ring cut along a periphery of the bed except for an area of the hinge cut formed by scanning the laser scan line along a circumference of the bed, wherein the ring cut covers all areas of the bed not covered by the bed cut.

18. The system of claim 17, wherein all cuts that form the corneal flap are performed at a same laser pulse energy

19. The system of claim 17, wherein the side cut includes a plurality of side cut layers formed in a sequence, each side cut layer extending within a depth range relative to the anterior corneal surface, wherein all except one of the plurality of side cut layers are located entirely within the cornea without reaching the anterior corneal surface, and wherein the plurality of side cut layers are aligned with each other and connect with each other to form the side cut.

20. The system of claim 17, wherein the controller is further programmed to control the scan line rotator, the XY-scanner and the Z-scanner to scan the laser scan line in the cornea to form a pocket cut prior to forming the bed cut,
wherein the pocket cut extends from a second depth posterior to the bed to the depth of the bed, wherein the bed cut is connected to the pocket cut;
wherein the pocket cut includes a ramp region and a pocket region connected to each other, wherein the pocket region is located at the second depth, and wherein the ramp region extends between the first depth and the second depth and is connected to both the bed and the pocket region.
